(19) United States
(12) Patent Application Publication
(10) Pub. No.: US 2014/0163099 A1
(43) Pub. Date: Jun. 12, 2014

(54) ANTIMICROBIAL COMPOSITION

(71) Applicant: TAKASAGO INTERNATIONAL CORPORATION, Tokyo (JP)

(72) Inventors: Kenya ISHIDA, Hiratsuka-shi (JP); Tomoko YAMAMOTO, Hiratsuka-shi (JP); Miho SUZUKI, Hiratsuka-shi (JP)

(73) Assignee: TAKASAGO INTERNATIONAL CORPORATION, Tokyo (JP)

(21) Appl. No.: 14/178,539

(22) Filed: Feb. 12, 2014

Related U.S. Application Data
(62) Division of application No. 13/537,971, filed on Jun. 29, 2012.
(60) Provisional application No. 61/506,273, filed on Jul. 11, 2011.

(30) Foreign Application Priority Data
Jun. 30, 2011 (JP) 2011-146348

(51) Int. Cl. A01N 37/36; A01N 37/18

(52) U.S. Cl. CPC A01N 37/36; A01N 37/18
USPC 514/546

(57) ABSTRACT

The objective of the present invention is to provide an antimicrobial composition that can be safely used with a broad range of foods and beverages, cosmetics, drugs, and other similar products while showing growth inhibition activity against a wide variety of microorganisms. The present invention relates to the antimicrobial composition comprising at least one cooling agent selected from the group consisting of menthyl 3-hydroxybutanoate, 2-methyl-3-(menthoxy)propane-1,2-diol, 2-(menthoxy)ethanol, 3-menthoxypropan-1-ol, 2-(2-menthoxyethoxy)ethanol, and menthyl glyoxylate.

### CROSS-REFERENCE TO RELATED APPLICATIONS

This is a divisional application of U.S. patent application Ser. No. 13/537,971 (pending) filed Jun. 29, 2012, which is a Non-Provisional of U.S. Provisional Application No. 61/506,273 filed Jul. 11, 2011, which claims priority based on Japanese Patent Application No. 2011-146348 filed Jun. 30, 2011, the contents of all of which are incorporated herein by reference in their entirety.

### TECHNICAL FIELD

The present invention relates to a safer antimicrobial composition which possesses excellent antimicrobial activity. Furthermore, the present invention also relates to a product comprising said the antimicrobial composition.

### BACKGROUND

Traditionally, various antimicrobial agents, such as natural and synthetic antimicrobial agents, are utilized in order to improve the preservation of food and cosmetic products. Benzoates, sorbates and so on are commercialized as synthetic antimicrobial agents. However, in the case where synthetic antimicrobial agents are used in products, such as food and cosmetics, which are in direct contact with the human body, it has been pointed out that further assurance of the safety of the antimicrobial agent is needed. On the other hand, it has been pointed out that a steady supply of natural antimicrobial agents cannot be ensured and cost a lot of money to produce.

Thus, it is desirable to obtain an antimicrobial agent which is safe and readily available, as well as easy to use with foods, cosmetics and so on when compared to common antimicrobial agents. In addition, as noted in the following document, work is being conducted with various antimicrobial agents to improve the shelf life of foods, cosmetics and so on.

JP 2007-45754A describes Macaranga extract extracted with a solvent such as an organic solvent as the natural antimicrobial agent.

JP 2011-06346A describes hydroxyundecanoic acid and a salt thereof produced by chemical synthesis as the synthetic antimicrobial agent.

JP 2004-18470A describes the art of using fragrance ingredient etc of foods as such mint oil which consists primarily of menthol as an antimicrobial agent.

WO 2011/050871A describes an antimicrobial agent in which an active ingredient is menthoxypropan diol produced by chemical synthesis.

### SUMMARY OF INVENTION

Accordingly, an objective of the present invention is to provide an antimicrobial composition that can be safely used with a broad range of foods and beverages, cosmetics, drugs, and other similar products while showing growth inhibition activity against a wide variety of microorganisms.

As a result of extensive studies aimed at solving the above problems, the present inventors have found that specific groups of cooling agents among the many compounds which are commonly known as cooling agents have remarkable antimicrobial activity which a person skilled in the art cannot predict, and the inventors have achieved the present invention. In addition, the compounds used as antimicrobial agents in the present invention have been used as cooling agents for long time, and thus the safety of the compounds in humans has been confirmed.

The present invention provides an antimicrobial composition comprising at least one cooling agent selected from the group consisting of menthyl 3-hydroxybutanoate, 2-methyl-3-(menthoxy)propane-1,2-diol, 2-(menthoxy)ethanol, 3-menthoxypropan-1-ol, 2-(2-menthoxyethoxy)ethanol, and menthyl glyoxylate.

Moreover, the present invention provides a flavor and/or fragrance composition comprising the above antimicrobial composition.

Moreover, the present invention provides a food and beverage, a cosmetic, a grocery good, a composition for oral or a drug comprising the above antimicrobial composition or the above flavor and/or fragrance composition.

Moreover, the present invention provides a method for producing a flavor and/or fragrance composition, a food and beverage, a cosmetic, a grocery good, a composition for oral or a drug, comprising the step of blending the antimicrobial composition with the food and beverage, the cosmetic, the grocery good, the composition for oral or the drug.

In addition, the term “cooling agent” in the present claims as used herein comprehends a dextrorotatory isomer and a levorotatory isomer, as well as a stereoisomeric mixture thereof.

The present invention can provide an antimicrobial composition and a flavor and/or fragrance composition which can have a broad range of safe applications with foods, cosmetics and drugs, and show a growth inhibition activity against a wide variety of microorganisms.

Furthermore, the present invention can provide a product, comprising said antimicrobial composition or said flavor and/or fragrance composition, in which the growth of microorganisms can be inhibited.

### DESCRIPTION OF EMBODIMENTS

Hereafter, each component used in the present antimicrobial composition will be described in detail.

<Antimicrobial Composition>

The antimicrobial composition of the present invention comprises at least one cooling agent selected from the group consisting of menthyl 3-hydroxybutanoate, 2-methyl-3-(1-menthoxy)propane-1,2-diol, 2-(menthoxy)ethanol, 3-menthoxypropan-1-ol, 2-(2-menthoxyethoxy)ethanol, and menthyl glyoxylate (hereafter, the cooling agent in the present composition is sometime referred to as “the present cooling agent”).

The present cooling agent has an unexpected remarkable antimicrobial activity. For example, when compared to a cooling agent other than the present cooling agent (e.g. N-alkyl-p-menthane-3-carboxamide, N-methyl-2-isopropyl-2,3-dimethylbutanamide, menthol, 3-menthoxypropane-1,2-diol and p-menthane-3,8-diol etc.), it can be understood that the present cooling agent has a extraordinary antimicrobial activity.

Although the all present cooling agents can have growth inhibition activities against both aerobic and anaerobic microorganisms (e.g. bacteria and fungi, etc.), menthyl 3-hydroxybutanoate and 3-menthoxypropan-1-ol have specifically high growth inhibition activities and potent growth inhibition activities against both aerobic and anaerobic microorganisms. Examples of microorganisms include Corynebacterium, Actinomyces and so on. Menthyl 3-hydroxybutanoate and 3-menthoxypropan-1-ol have 2 to 8 times as much growth inhibition activity as 3-menthoxypropane-1,2-diol (which is known as an antimicrobial agent), and can have more than or equal to 8 times as much growth inhibition activity as 3-menthoxypropane-1,2-diol, depending on kinds of target microbe species.

Furthermore, when compared 2-methyl-3-(menthoxy)propane-1,2-diol with 3-menthoxypropane-1,2-diol (which is known as an antimicrobial agent), the former has about 2 times as much growth inhibition activity as the latter, depending on kinds of target microbe species.

Therefore, it is further preferable that the present invention comprises at least one cooling agent selected from the group consisting of menthyl 3-hydroxybutanoate, 2-methyl-3-(1-menthoxy)propane-1,2-diol, and 3-menthoxypropan-1-ol.

Furthermore, it is preferable that menthyl 3-hydroxybutanoate is used as the antimicrobial agent against Porphyromonas such as Porphyromonas gingivalis (Periodontal pathogen), Staphylococcus such as Staphylococcus aureus (skin flora) and Corynebacterium such as Corynebacterium xerosis (armpit odor-causing bacteria), and it is preferable that 2-methyl-3-(1-menthoxy)propane-1,2-diol is used as the antimicrobial agent against Staphylococcus such as Staphylococcus aureus (skin flora).

Furthermore, 2-(menthoxy)ethanol and 2-(2-menthoxyethoxy)ethanol have the growth inhibition activity against anaerobic microorganisms specially, further specifically 2-(menthoxy)ethanol and 2-(2-menthoxyethoxy)ethanol have more than 2 times as much growth inhibition activity against Propionibacterium acnes (JCM6473) and (ATCC6919) as well as Fusobacterium nucleatum as 3-menthoxypropane-1,2-diol (which is known as an antimicrobial agent).

Furthermore, menthyl glyoxylate has 2 to 16 times or more than 16 times as much growth inhibition activity as 3-menthoxypropane-1,2-diol (which is known as an antimicrobial agent), depending on kinds of target microbe species.

The above cooling agents are readily commercially available, and can be synthesized by common methods.

Furthermore, it is preferable that the present antimicrobial composition further comprises at least one component selected from the group consisting of menthol, isopulegol, menthone, camphor, pulegol, cineol, mint oil, N-alkyl-p-menthane-3-carboxamide, p-menthane-3,8-diol, 4-1-menthoxybutane-1-ol, 1-(2-hydroxy-4-methyl-cyclohexyl)-ethanone, menthyl lactate, menthol glycerol ketal, N-methyl-2-isopropyl-2,3-dimethylbutanamide, menthyl succinate, menthyl glutarate, peppermint oil, eucalyptus oil, spearmint oil, vanillyl ethyl ether, vanillyl propyl ether, vanillin propylene glycol acetal, ethyl vanillin propylene glycol acetal, capsaicin, gingerol, vanillyl butyl ether, 4-(1-menthoxy-methyl)-2-phenyl-1,3-dioxolane, 4-(1-menthoxy-methyl)-2-(3′,4′-dihydroxy-phenyl)-1,3-dioxolane, 4-(1-menthoxy methyl)-2-(2′-hydroxy-3′-methoxy-phenyl)-1,3-dioxolane, 4-(1-menthoxy-methyl)-2-(4′-methoxyphenyl)-1,3-dioxolane, 4-(1-menthoxy-methyl)-2-(3′,4′-methylenedioxy-phenyl)-1,3-dioxolane, 4-(1-methoxy-methyl)-2-(3′-methoxy-4′-hydroxyphenyl)-1,3-dioxolane, red pepper oil, red pepper oleoresin, vanillylamide nonylate, jambu oleoresin, Japanese pepper extract, sanshool-I, sanshool-II, sanshoamide, black pepper extract, chavicine, piperine and spilanthol (hereafter, the component comprised in this group is sometime referred to as “combined component”). It is expected that in the present antimicrobial composition, the combined component will further enhance the antimicrobial activity of the present antimicrobial composition, as well as that the combined component will be able to provide and control pleasant cooling and heating sensations.

Furthermore, it is also possible to provide the desired antimicrobial activity synergistically by using the present cooling agent in combination with the following compound which is used widely as a typical antimicrobial agent: paraben, benzoic acids (e.g. benzoic acid, sodium benzoate etc.), salicylic acids, 1,2-alkane diol, ester or ether of hydroxycarboxylic acid, benzalkonium chloride, benzethonium chloride, lysozyme chloride, halocarban, trichlorocarbanilide, chlorhexidine hydrochloride, isopropyl methyl phenol, aluminum phenol sulfonate, triclosan, chlorhexidine gluconate, a solution of sodium hydrogen sulfite, sodium sulfite (crystal), (anhydrous) sodium sulfite, Udo extract, Styrax japonica extract, Artemisia capillaris extract, enzyme-degradable hatomugi (Adlay) extract, Sodium hyposulfite, Milt protein extract, sorbic acid, potassium sorbate, thujaplicin (extract), sodium dehydroacetate, sulfur dioxide, isobutyl paraoxybenzoate, isopropyl paraoxybenzoate, ethyl paraoxybenzoate, butyl paraoxybenzoate, propyl paraoxybenzoate, potassium pyrosulfite, sodium pyrosulfitesodium, propionic acid, calcium propionate, sodium propionate, pectin degradation products, Magnolia obovata extract, ε-polylysine, forsythia extract, allspice oil, origanum oil, orange oil, cassia oil, cumin oil, clove oil, coriander oil, perilla oil, citronella oil, cinnamon oil, calamus oil, sage oil, geranium oil, thyme oil, dill oil, tree moss oil, Thujopsis dolabrata oil, pimento oil, fennel oil, bay oil, vetiver oil, penny royal oil, Peru balsam oil, eucalyptus oil, lavender oil, lemongrass oil, lemon oil, rosemary oil, rose oil, laurel oil, Thujopsis dolabrata oil, anise oil, wintergreen oil, estoragon oil, onion oil, cardamom oil, caraway oil, cade oil, star anise oil, cedarwood oil, celery oil, tarragon oil, Formosan cypress oil, nutmeg oil, birch oil, black pepper oil, basil oil, white pepper, marjoram oil, mace oil, cananga oil, jasmine oil, spearmint oil, peppermint oil, patchouli oil, bois de rose oil, green tea extract, oolong tea extract, tea catechins, ginger oil, cereli seed oil, lovage oil, valerian oil, hop oil, ambrette seed oil, dill seed oil, pennyroyal oil, tansy oil, winelees oil, ephedra herb extract, Zanthoxylum piperitum oil, ophiopogon tuber extract.

<<Evaluation of the Antimicrobial Activity>>

In the present invention, the growth inhibition activity, which is an indicator of the antimicrobial activity, is evaluated by determining the Minimum Inhibitory Concentration (MIC) with an agar or liquid medium. In addition, although it cannot be determined if the microbicidal or bacteriostatic activity contributes to the growth inhibition activity, in the Determining the Minimum Inhibitory Concentration in the present specification, since antimicrobial activity can include microbicidal and bacteriostatic activities, the above evaluation does not matter.

<Flavor and/or Fragrance Composition>

The present flavor and/or fragrance composition composes the above mentioned antimicrobial composition. Although the content of the present cooling agent in the flavor and/or fragrance composition can be arbitrarily regulated depending on the dosage level of the flavor and/or fragrance composition, the content is generally 0.01 to 50% by weight, preferably 0.01 to 20% by weight, further preferably 0.05 to 10% by weight, based on the total mass of the flavor and/or fragrance composition. When the content is more than 50% by weight, there are few economic advantages, while when the content is less than 0.01% by weight, the effect caused by the present flavor and/or fragrance composition may be not sufficiently displayed.

The present flavor and/or fragrance composition can further comprise any components to the extent not to spoil the antimicrobial activity, which, include (but not limited to) natural essential oils, synthetic essential oils, citrus oils, animal perfume.

The synthetic aromachemicals used in the present flavor and/or fragrance composition are not particularly limited as long as they have been commonly used in flavors and fragrances industry, and examples of the which include at least one selected the group consisting of esters, alcohols, aldehydes, ketones, phenols, ethers, lactones, hydrocarbons, nitrogen-containing compounds, sulfur-containing compounds and acids described in “Synthetic flavoring ingredient chemicals and product knowledge” (Genichi Indo, Chemical Daily Co., Ltd.) etc.

Examples of the esters include, propyl formate, butyl formate, amyl formate, octyl formate, linalyl formate, citronellyl formate, geranyl formate, neryl formate, terpinyl formate, ethyl acetate, isopropyl acetate, isoamyl acetate, hexyl acetate, cis-3-hexenyl acetate, trans-2-hexenyl acetate, octyl acetate, nonyl acetate, decyl acetate, dodecyl acetate, dimethyl undecadienyl acetate, styrallyl acetate, ocimenyl acetate, myrcenyl acetate, dihydro myrcenyl acetate, linalyl acetate, citronellyl acetate, geranyl acetate, neryl acetate, tetrahydro-mugol acetate, lavandulyl acetate, nerolidol acetate, dihydrocuminyl acetate, terpinyl acetate, citril acetate, nopyl acetate, dihydroterpinyl acetate, 2,4-dimethyl-3-cyclohexenyl methyl acetate, myraldyl acetate, veticol acetate, decenyl propionate, linalyl propionate, geranyl propionate, neryl propionate, terpinyl propionate, tricyclodecenyl propionate, styrallyl propionate, anisil propionate, octyl butyrate, neryl butyrate, cinnamyl butyrate, isopropyl isobutyrate, octyl isobutyrate, linalyl isobutyrate, neryl isobutyrate, linalyl isovalerate, terpinyl isovalerate, phenylethyl isovalerate, 2-methylpentyl 2-methylvalerate, methyl 3-hydroxyhexanoate, ethyl 3-hydroxyhexanoate, methyl octanoate, octyl octanoate, linalyl octanoate, methyl nonanoate, methyl undecylenate, linalyl benzoate, methyl cinnamate, isoprenyl angelicate, methyl gelanate, triethyl citrate, ethyl acetoacetate, ethyl 2-hexylacetoacetate, ethyl benzyl-acetoacetate, ally 2-ethylbutyrate, ethyl 3-hyoxybutyrate, ethyl nonanoate, ethyl decanoate, ethyl 2,4-decadienoate, propyl 2,4-decadienoate, methyl anthranylate, ethyl N-methyl-anthranylate, and the like.

Examples of the alcohols include, 3-heptanol, 1-nonanol, 1-undecanol, 2-undecanol, 1-dodecanol, prenol, 10-undecen-1-ol, dihydrolinalool, tetrahydromugol, myrcenol, dihydromyrcenol, tetrahydromyrcenol, ocimenol, terpineol, hotrienol, 3-thujanol, benzylalcohol, β-phenylethyl alcohol, α-phenylethyl alcohol, 3-methyl-1-pentanol, 1-heptanol, 2-heptanol, 3-octanol, 1-nonanol, 2-nonanol, 2,6-dimethyl-heptanol, 1-decanol, trans-2-hexenol, cis-4-hexenol, methyltrimethylcyclopentenylbutenol, citronellol, dihydro-myrcenol, rhodinol, geraniol, nerol, linalool, tetrahydrolinalool, dimethyloctanol, hydroxycitronellol, isopulegol, menthol, terpineol, dihydro-terpineol, carveol, dihydro-carveol, perilla alcohol, 4-thujanol, myrtenol, β-fenchyl alcohol, farnesol, nerolidol, cedrenol, anisealcohol, hydratropic alcohol, 3-phenyl-propyl alcohol, cinnamic alcohol, amylcinnamic alcohol, and the like.

Examples of the aldehydes include, acetaldehyde, n-hexanal, n-heptanal, n-octanal, n-nonanal, 2-methyloctanal, 3,5,5-trimethylhexanal, decanal, undecanal, 2-methyldecanal, dodecanal, tridecanal, tetradecanal, trans-2-hexenal, trans-4-decenal, cis-4-decenal, trans-2-decenal, 10-undecenal, trans-2-undecenal, trans-2-dodecenal, 3-dodecenal, trans-2-tridecenal, 2,4-hexadienal, 2,4-decadienal, 2,4-dodecadienal, 5,9-dimethyl-4,8-decadienal, citral, dimethyloctanal, α-methylene citronellal, citronellyl oxyacetaldehyde, myrtenal, neral, α- or β-sinensal, myrac aldehyde, phenyl acetoaldehyde, octanal dimethyl acetal, nonanal dimethyl acetal, decanal dimethyl acetal, decanal diethyl acetal, 2-methyl undecanal dimethyl acetal, citral dimethyl acetal, citral diethyl acetal, citral propylene glycol acetal, n-valeraldehyde, iso-valeraldehyde, 2-methyl butanal, 2-pentenal, trans-2-heptenal, trans-2-nonenal, 2,6-dimethyl-5-heptenal, 2,4-undecadienal, trimethyl decadienal, citronellal, hydroxyl citronellal, safranal, vernaldehyde, benzaldehyde, p-isopropyl-phenyl acetoaldehyde, p-methyl-hydro-tropaldehyde, phenyl propionaldehyde, 2-methyl-3-(4-methyl phenyl)-propanal, cycramen aldehyde, cinnamic aldehdye, salicylaldehyde, anisaldehyde, p-methyl-phenoxyacetaldehyde, acetaldehyde diethyl acetal, citronellyl methyl acetal, acetaldehyde 2-phenyl-2,4-pentandiol acetal, 2-hexenal diethyl acetal, cis-3-hexenal diethyl acetal, heptanal diethyl acetal, 2-hexyl-5-methyl-1,3-dioxolane, citronellal-cyclo-mono-glycol acetal, hydroxyl-citronellal dimethyl acetal, phenyl acetaldehyde dimethyl acetal, and the like.

Examples of the ketones include, 2-pentanone, 3-hexanone, 2-heptanone, 3-heptanone, 4-heptanone, 2-octanone, 3-octanone, 2-nonanone, 2-undecanone, methyl heptenone, dimethyl octenone, geranyl acetone, farnesyl acetone, 2,3,5-trimethyl-4-cyclohexenyl-1-methyl ketone, nerone, nootkatone, dihydronootkatone, acetophenone, 4,7-dihydro-2-isopentyl-2-methyl-1,3-dioxepin, 2-pentanone, 3-hexanone, 2-heptanone, 2,3-hexanedione, 3-nonanone, ethyl isoamyl ketone, diacetyl, amyl-cyclopentenone, 2-cyclopentyl cyclopentanone, hexyl cyclopentanone, heptyl cyclopentanone, cis-jasmone, dihydro-jasmone, trimethyl pentyl cyclopentanone, 2-(2-(4-methyl)-3-cyclohexanone-1-yl)-propyl-cyclopentanone, damascone, α-dynascone, trimethyl cyclohexenyl butenone, jonan (ionone), methylionone, allylionone, pricatone, cashmeran, L-carvone, menthone, camphor, p-methyl acetophenone, p-methoxy-acetophenone, benzylidene acetone, raspberry ketone, methyl naphtyl ketone, benzophenone, furfural acetone, homofuronol, maltol, ethyl maltol, acetoacetic acid ethyl ethyleneglycol ketal, and the like.

Examples of the phenols include, thymol, carvacrol, β-naphthol isobutyl ether, anethole, β-naphthol methyl ether, β-naphthol ethyl ether, creosol, veratrole, hydroquinone dimethyl ether, 2,6-dimethoxyl phenol, 4-ethyl guaiacol, eugenol, isoeugenol, ethyl isoeugenol, tert-butyl hydroquinone dimethyl ether, and the like.

Examples of the ethers include, decyl vinyl ether, α-terpinyl methyl ether, iso-proxen, 2,2-dimethyl-5-(1-methyl-1-propenyl)-tetrahydro-furan, rosefuran, 1,4-cineol, nerol oxide, 2,2,6-trimethyl-6-vinyl tetrahydro-pyran, methyl hexyl ether, ocimene epoxide, limonene oxide, rhubofix, caryophyllene oxide, linalool oxide, 5-isopropenyl-2-methyl-2-vinyl tetrahydro-furan, nerol oxide, rose oxide, and the like.

Examples of the lactones include, γ-undecalactone, δ-dodecalactone, γ-hexylactone, γ-nonalactone, γ-decalactone, γ-dodecalactone, jasmine lactone, methyl γ-decalactone, 7-decenolactone, jasmolactone, propylidene phthalide, δ-hexylactone, δ-2-decenolactone, ε-dodecalactone, dihydrocoumarin, coumarin, and the like.

Examples of the hydrocarbons include, ocimene, limonene, α-phellandrene, terpinene, 3-caren, bisabolene, valencene, alloocimene, myrcene, farnesene, α-pynene, β-pynene, camphene, terpinolene, p-cymene, cedrene, β-caryophyllene, cadinene, and the like.

Examples of the compounds containing nitrogen or sulfur include, methyl anthranilate, ethyl anthranilate, methyl N-methyl-anthranilate, methyl N-2′-methyl-pentylidyne-anthranilate, ligantral, dodecane nitrile, 2-tridecene nitrile, geranyl nitrile, citronellyl nitrile, 3,7-dimethyl-2,6-nonadieno nitrile, indole, 5-methyl-3-heptanone oxime, limonene thiol, 1-P-menthen-8-thiol, butyl anthranilate, cis-3-hexenyl anthranilate, phenyl ethyl anthranilate, cinnamyl anthranilate, dimethyl sulfide, 8-mercaptomenthone, and the like.

Examples of the acids include, acetic acid, propionic acid, butyric acid, valeric acid, hexanoic acid, octanoic acid, decanoic acid, dodecanoic acid, 2-decenoic acid, geranoic acid, 2-methyl-butyric acid, 2-ethyl-butyric acid, phenyl-acetic acid, cinnamic acid, iso-butyric acid, iso-valeric acid, 3-methyl valeric acid, 2-hexenoic acid, 2-methyl-2-pentenoic acid, 2-methyl-heptanoic acid, myristic acid, stearic acid, lactic acid, pyruvic acid, cyclohexane-carbonic acid, and the like.

Examples of the natural fragrances include, sweet orange, bitter orange, neroli, mandarin, petit grain, bergamot, tangerine, onshuu mandarin orange, daidai (Citrus aurantium), hassaku (Citrus hassaku), iyokan (Citrus Iyo), lemon, lime, grape fruit, yuzu (Citrus junos), sudachi (Citrus sudachi), cabosu (Citrus sphaerocarpa), sweety and the like.

In addition, the following can also be used other than the above natural fragrances as aroma and flavoring substance or improving agent of the aroma and flavoring; citronella, elemi, olibanum, marjoram, angelica root, star anise, basil, hay, calamos, caraway, cardamom, pepper, cascarilla, ginger, sage, clary sage, clove, coriander, eucalyptus, fennel, pimento, juniper, fenugreek, laurel, mace, sugi (cedar), senkyu, almond, apple mint, anise, artemisia, alfalfa, apricot, ambrette, rush, strawberry, fig, ylang-ylang, winter green, ume apricot, elder, enju (Japanese-pagota tree), oak moss, allspice, orris, currant, cassie, chamomile, galanga, Chinese quince, gambir, guava, gooseberry, camphor tree, gardenia, cubeb, cumin, cranberry, cola, japanese pepper, sandarac, sandal wood, red sandal wood, perilla, civet, jasmine, ginger, ginseng, cinnamon, starfruit, styrax, spearmint, geranium, thyme, davana, tansy, tangerine, champac, tuberose, camellia, dittany, tolu balsam, tonka, nut, jujube, nutmeg, nanten, ti-tree, carrot, violet, pineapple, hibiscus, honey, mint, passion fruit, vanilla, rose, hyssop, hinoki, fusel oil, buchu, peppermint, pepino, verbena, bois de rose, popaw, boldo, boronia, pine, mango, bees wax, mimosa, milfoil, musk, maple, melissa, melon, peach, yara-yara, lavender, liqueur, litsea, linden, rue, water apple, rosemary, lovage, and the like.

These contents of these components for blending perfume are not specially limited, and can be selected according to the purpose and use (application) arbitrary.

Further, the present flavor and/or fragrance composition can be further blended with other materials depending on purposes and applications. Particular examples include citrus flavor such as orange, lemon, lime, grape fruit, yuzu (Citrus junos) and sudachi (Citrus sudachi); berry flavor such as strawberry, raspberry, blue berry; tropical fruit flavor such as mango, papaya, guava, passion fruit, litchi; fruit flavor such as apple, grape, pineapple, banana, peach, melon, apricot, ume (Prunus mume), and cherry; tea and coffee flavor such as green tea, oolong tea, black tea, and coffee; meat flavor such as beef, pork, and chicken; herb and spice flavor such as asafetida, ajowan, anise, angelica, fennel, allspice, cinnamon, cassia, chamomile, mustard, cardamom, caraway, cumin, clove, pepper, coriander, sassafras, savory, japanese pepper, perilla, juniperberry, ginger, star anise, horseradish, sage, thyme, tarragon, dill, capsicum, jujube, nutmeg, basil, parsley, marjoram, rosemary, laurel, and wasabi (Wasabia japonica); vegetable flavor such as onion, garlic, green onion, cabbage, carrot, celery, shiitake (Lentinula edodes (Berk.) Pegler), matsutake (Tricholoma matsutake), tomato, burdock, and honewort; mint flavor such as peppermint, spearmint, and Japanese mint; vanilla flavor; nut flavor such as almond, cashew nut, peanut, hazel nut, walnut, chestnut, macadamia nut, pecan nut, pistachio, Brazil nut, and coconut; liqueur flavor such as wine, whisky, brandy, rum, gin and liqueur; grain flavor such as corn, potato, sweet potato, cooked rice, and bread; sugar flavor such as honey, maple syrup, sugar, brown sugar and molasses.

The synthetic aromachemicals are readily commercially available, and easily synthesized if necessary. Also, the natural materials are readily commercially available, and easily obtained with common methods like extraction/purification.

Dosage level of the present composition can be adjusted depending upon the application for the designated products.

Furthermore, the formulation (e.g., liquid, solid, poweder, gel, mist, aerosol and so on) can be selected based on the character of designated products.

<Food and Beverage, Cosmetic, Grocery Good, Composition for Oral, or Drug>

The present food and beverage, cosmetic, grocery good, composition for oral or drug (hereafter, these are sometime referred to as “product(s)” collectively) comprises the above antimicrobial composition or the above flavor and/or fragrance composition. Although the content of the present cooling agent in the products can be arbitrarily regulated depending on the dosage level of these products, the content is generally 0.0001 to 30% by weight, preferably 0.001 to 20% by weight, further preferably 0.005 to 10% by weight, based on the total mass of the products. When the content is more than 30% by weight, there are few economic advantages, while when the content is less than 0.0001% by weight, the effect caused by the present product may be not sufficiently displayed.

Moreover, the sum of the contents of cooling agent, combined component, and common antimicrobial agent can be generally set to for example 0.01 to 50% by weight according to the total mass of the product.

Examples of the present food and beverage include, drinks such as fruit juice drinks, fruit wines, milk drinks, carbonated drink, soft drink and drink preparations; ices such as ice creams, sherbets and ice candies; desserts such as jelly and pudding; Western style cakes such as cake, cookie, chocolate and chewing gum, Japanese style confections such as bean-jam bun, sweet beans jelly and Uiro; jams; candies; breads; tea drinks or luxury drinks such as green tea, Oolong tea, black tea, persimmon leaf tea, chamomile tea, low striped bamboo tea, mulberry tea, dokudami tea, Pu-erh tea, mate tea, Rooibos tea, Gymnema tea, Guava tea, coffee and cocoa; soups such as Japanese style soup, Western style soup and Chinese soup; flavoring and seasoning; various instant drinks or convenience foods; various snack foods and the like.

Examples of the present cosmetic include, fragrance products (perfume, eau de perfume, eau de toilette, eau de cologne and the like), basic cosmetics (cleansing cream, banishing cream, cleansing cream, cold cream, massage cream, milky lotion, skin lotion, beauty lotion, pack, makeup remover and the like), finishing cosmetics (foundation, face powder, solid face powder, talcum powder, rouge, lip barm, cheek rouge, eye liner, mascara, eye shadow, eyebrow pencil, eye pack, nail enamel, enamel remover and the like), hair cosmetics (pomade, brilliantine, set lotion, hair stick, hair solid, hair oil, hair treatment, hair cream, hair tonic, hair liquid, hair spray, bandrine, revitalizing hair tonic, hair dye and the like), suntan cosmetics (suntan products, sunscreen product and the like), medicated cosmetics (antiperspirant, after shaving lotion or gel, permanent wave agent, medicated soap, medicated shampoo, medicated skin cosmetics and the like).

Additionally, Examples of the present grocery good include deodorant and air freshener (solid type, gel type, such as the liquid type), tissue paper, toilet paper and the like.

Additionally, Examples of the present composition for oral include oral goods (dentifrice, oral cavity cleaner, mouth wash, troche, chewing gums and the like) and the like.

Additionally, Example of the present drug include skin external preparations such as poultices and ointment, internal medicines and the like.

### EXAMPLES

Hereafter, the present invention will be described in more detail with reference to the following Examples, which do not limit the present invention.

Regarding substance names used in the following Examples and Comparative examples, “Coolact®” is the registered trademark of TAKASAGO INTERNATIONAL CORPORATION, and “WS” is the registered trademark of Millennium specialty chemicals. This “Coolact®” is also referred to as “CA” herein. The IUPAC names of these substances correspond to the designations as the following Table 1.

TABLE 1

| Cooling agent | Designation |
|---|---|
| [Example 1] | |
| menthyl 3-hydroxybutanoate | CA20 |
| 2-methyl-3-(menthoxy)propane-1,2-diol | CA1 |
| 2-(menthoxy)ethanol | CA5 |
| 3-menthoxypropan-1-ol | CA15 |
| 2-(2-menthoxyethoxy)ethanol | CA55 |
| menthyl glyoxylate | CA MGH |
| [Comparative Example 1] | |
| N-alkyl-p-menthane-3-carboxamide | WS-3 |
| N-methyl-2-isopropyl-2,3-dimethylbutanamide | WS-23 |
| menthol | 1-menthol |
| 3-menthoxypropane-1,2-diol | CA10 |
| p-menthane-3,8-diol | CA38 |

CA: Coolact

In addition, the above menthol was purchased from TAKASAGO INTERNATIONAL CORPORATION.

Hereafter, the substance names is expressed using the designations.

### Example 1

### Measurement of Growth Inhibition Activity Against Aerobic Bacteria

In the test for the growth inhibition activity, the following strains were used as skin-related bacteria:
Staphylococcus epidermidis: Staphylococcus epidermidis (JCM2414T and ATCC 12228)
Staphylococcus aureus: Staphylococcus aureus (NBRC12732 and JCM2151)
Skin indigenous bacteria which introduce a superficial infection: Corynebacterium minutissimum (NBRC15361T)
Armpit odor-causing bacteria: Corynebacterium xerosis (JCM1324)

Furthermore, the following bacteria were also used as other general aerobic bacteria:
Escherichia coli: Escherichia coli (NBRC3972)
Pseudomonas aeruginosa: Pseudomonas aeruginosa (NBRC13275)
Bacteria in intestines: Proteus vulgaris (NBRC3167)
Pneumobacillus: Klebsiella pneumoniae (NBRC13277)
Bacillus subtilis: Bacillus subtilis (NBRC3134)

The test strains were cultured in the liquid medium which the Bioresource center recommends, and then the test strains were subcultured to a Mueller Hinton Broth (Difco) at 30 degrees C. The grown bacteria were diluted to 10⁶ CFU/ml with saline to adjust the bacteria fluids for inoculation.

Furthermore, in order to examine the growth inhibition activities against the bacteria by the above cooling agents, the concentration of the cooling agents: (3S)-CA20, (3R)-CA20, CA1, CA5, CA15, CA55 or CA MGH was diluted with ethanol to the required level of concentration for the determining the Minimum Inhibitory Concentration to produce the sample liquids.

Next, 100 μL of each sample liquid was added to 10 mL of a Mueller Hinton Agar (Difco) agar medium solution in a soluble state, and each agar medium solution was poured into a petri dish and solidified to prepare test plates having several concentrations of the sample liquids (several concentrations of the above cooling agents). Subsequently, 5 μL of the above bacteria fluids for inoculation were stamped on the test plates, and then the test plates were cultured at 37 degrees C. overnight.

In this way, the growth inhibition activities by (3S)-CA20, (3R)-CA20, CA1, CA5, CA15, CA55, CA MGH were examined. The minimum concentration of the cooling agent in which the growth of bacteria could not be confirmed was considered as the MIC (Minimum Inhibitory Concentration).

### Comparative Example 1

In a manner similar to Example 1, the effects of WS-3, WS-23, 1-menthol, CA10, and CA38 were examined. The minimum concentration of the cooling agent in which the grown strain could not be confirmed was considered as the MIC (Minimum Inhibitory Concentration).

The MICs of Example 1 and Comparative Example 1 are denoted in Table 2 (Unit: ppm).

TABLE 2

| | strain | | | | | |
|---|---|---|---|---|---|---|
| sample | JCM2414T | ATCC12228 | NBRC12732 | JCM2151 | NBRC15361T | JCM1324 |

Example 1

| | | | | | | |
|---|---|---|---|---|---|---|
| (3S)-CA20 | 100 | | 200 | 100 | 200 | 100 |
| (3R)-CA20 | 200 | | 200 | 100 | 200 | 100 |
| CA1 | 400 | 800 | 800 | 400 | 400 | 800 |
| CA5 | 800 | 800 | 800 | 400 | 800 | 800 |
| CA15 | 400 | 400 | 400 | 200 | 200 | 400 |
| CA55 | 800 | 800 | 800 | 800 | 800 | 800 |
| CA MGH | 50 | 50 | 100 | 100 | 100 | 100 |

Comparative Example 1

| | | | | | | |
|---|---|---|---|---|---|---|
| WS-3 | >1600 | | >1600 | >1600 | >1600 | >1600 |
| WS-23 | >1600 | | >1600 | >1600 | >1600 | >1600 |
| l-menthol | 1600 | 1600 | 1600 | 1600 | 1600 | 1600 |
| CA10 | 800 | 1600 | 1600 | 800 | 800 | 800 |
| CA38 | >1600 | >1600 | >1600 | >1600 | >1600 | >1600 |

| | strain | | | | |
|---|---|---|---|---|---|
| sample | NBRC3972 | NBRC13275 | NBRC3167 | NBRC13277 | NBRC3134 |
| | Example 1 | | | | |
| (3S)-CA20 | >1600 | >1600 | >1600 | 200 | 200 |
| (3R)-CA20 | >1600 | >1600 | >1600 | 200 | 200 |
| CA1 | >1600 | >1600 | >1600 | 400 | 400 |
| CA5 | >1600 | >1600 | 1600 | 400 | 800 |
| CA15 | >1600 | >1600 | >1600 | 200 | 400 |
| CA55 | >1600 | >1600 | >1600 | 800 | 800 |
| CA MGH | 800 | 800 | 100 | 50 | 50 |
| | Comparative Example 1 | | | | |
| WS-3 | >1600 | >1600 | >1600 | >1600 | >1600 |
| WS-23 | >1600 | >1600 | >1600 | >1600 | >1600 |
| l-menthol | 1600 | >1600 | 1600 | 1600 | 1600 |
| CA10 | >1600 | >1600 | 1600 | 800 | 800 |
| CA38 | >1600 | >1600 | >1600 | >1600 | 800 |

In view of the result in Table 2, it was understood that (3S)-CA20, (3R)-CA20, CA1, CA5, CA15, CA55, and CA MGH (the present Example) had low MIC values against many strains compared with WS-3, WS-23, 1-menthol, CA10 and CA38 (the Comparative Example). Thus, it was understood that the present cooling agent can provide antimicrobial activities against skin-related bacteria such as Staphylococcus aureus, and armpit odor-causing bacteria, and other aerobic bacteria.

### Example 2

### Measurement of Growth Inhibition Activity Against Anaerobic Bacteria

In the test for growth inhibition activity, the following strains were used:
Acne bacteria: Propionibacterium acnes (JCM6473) and (ATCC6919)
Abscess bacteria: Bacteroides fragilis (GAI5560)
Cavity-causing bacteria: Streptococcus mutans (JCM5175), Actinomyces naeslundii (JCM8350) and Actinomyces viscosus (JCM8352)
Periodontal pathogen: Fusobacterium nucleatum (JCM6328), Porphyromonas gingivalis (JCM8525), Prevotella nigrescens (JCM6322)

The test strains were cultured in a GAM medium (Nissui Pharmaceutical Co., LTD), a Schaedler Broth (BBL) or a Trypticase Soy Broth (BBL) liquid medium, and then the grown bacteria were diluted in saline to prepare turbidity 0.5 McFarland of the bacteria fluids for inoculation. The bacteria were cultured at 37 degrees C. for 3 days under an anaerobic condition by using AnaeroPack® from MGC.

Furthermore, in order to examine the growth inhibition activities against the bacteria by the above cooling agent, the concentration of the cooling agents: (3S)-CA20, (3R)-CA20, CA1, CA5, CA15, CA55 or CA MGH was diluted with ethanol to the required level of concentrations for the determining the Minimum Inhibitory Concentration to produce the sample liquids.

Next, 100 μL of each sample liquid was added to 10 mL of a Trypticase Soy Broth agar medium solution in a soluble state, and each agar medium solution was poured into a petri dish and solidified, to prepare the test plates. Subsequently, 5 μL of above the bacteria fluids for inoculation were stamped on the test plates, and then the test plates were cultured at 37 degrees C. for 3 days.

In this way, in a manner similar to Example 1, the growth inhibition activities by (3S)-CA20, (3R)-CA20, CA1, CA5, CA15, CA55, CA MGH were examined. The minimum concentration of the cooling agent in which the growth of bacteria could not be confirmed was considered as the MIC (Minimum Inhibitory Concentration). The concentrations in this time are denoted in Table 3 (Unit: ppm).

### Comparative Example 2

In a manner similar to Example 2, the effects of WS-3, WS-23, 1-menthol, CA10, and CA38 were examined. The minimum concentration of the cooling agent in which the growth of bacteria could not be confirmed was considered as the MIC (Minimum Inhibitory Concentration). The concentrations in this time are denoted in Table 3 (Unit: ppm).

TABLE 3

| | strain | | | | | | | | |
|---|---|---|---|---|---|---|---|---|---|
| sample | JCM6473 | ATCC6919 | GAI5560 | JCM5175 | JCM8350 | JCM8352 | JCM6328 | JCM8525 | JCM6322 |

Example 2

| | | | | | | | | | |
|---|---|---|---|---|---|---|---|---|---|
| (3S)-CA20 | 200 | 200 | 100 | 200 | 100 | 100 | | 50> | 100 |
| (3R)-CA20 | 200 | 400 | 100 | 200 | 200 | 100 | | 100 | 200 |
| CA1 | 400 | 400 | 100 | 800 | 800 | 50> | 800 | 200 | 200 |
| CA5 | 400 | 400 | 200 | 800 | 800 | 200 | 400 | | 200 |
| CA15 | 200 | 400 | 100 | 400 | 400 | 200 | 400 | | 100 |
| CA55 | 800 | 800 | 100 | 800 | 800 | 800 | 800 | | 100 |
| CA MGH | 200 | 100 | 50 | 100 | 200 | 200 | 100 | 50 | |

Comparative Example 2

| | | | | | | | | | |
|---|---|---|---|---|---|---|---|---|---|
| WS-3 | >1600 | >1600 | >1600 | >1600 | >1600 | >1600 | | 200 | >1600 |
| WS-23 | >1600 | >1600 | >1600 | >1600 | >1600 | >1600 | | >1600 | >1600 |
| l-menthol | 1600 | 1600 | 1600 | >1600 | 1600 | 1600 | 1600 | | 1600 |
| CA10 | 800 | 800 | 100 | 800 | 800 | 50> | 800 | | 200 |
| CA38 | >1600 | >1600 | 1600 | >1600 | >1600 | >1600 | >1600 | | 1600 |

In view of the result in Table 3, it was understood that (3S)-CA20, (3R)-CA20, CA1, CA5, CA15, CA55, and CA MGH (the present Example) had low MIC values against many strains compared with WS-3, WS-23, 1-menthol and CA38 (the Comparative Example). The former (the present Example) also had low MIC values compared with CA 10. Thus, it was understood that the present cooling agent can provide the antimicrobial activity against anaerobic bacteria such as acne bacteria, abscess bacteria, cavity-causing bacteria, and Periodontal pathogen.

Although the present invention has been described and illustrated in detail, it is clearly understood that the same is by way of illustration and example only and is not to be taken by way of limitation, the spirit and scope of the present invention being limited only by the terms of the appended claims.

1. A method for inhibiting a growth of microbe in a flavor and/or fragrance composition, comprising adding menthyl 3-hydroxybutanoate to the flavor and/or fragrance composition.

2. The method for inhibiting a growth of microbe according to claim 1, wherein the menthyl 3-hydroxybutanoate content is 0.01 to 50% by weight based on the total amount of the flavor and/or fragrance composition.

3. The method for inhibiting a growth of microbe according to claim 1, comprising adding menthyl 3-hydroxybutanoate and spilanthol to the flavor and/or fragrance composition.

4. The method for inhibiting a growth of microbe according to claim 3, wherein the menthyl 3-hydroxybutanoate content is 0.01 to 50% by weight based on the total amount of the flavor and/or fragrance composition.

5. A method for inhibiting a growth of microbe in a food and beverage, a cosmetic, a grocery good, a composition for oral administration or a drug, comprising adding menthyl 3-hydroxybutanoate to the food and beverage, the cosmetic, the grocery good, the composition for oral administration or the drug.

6. The method for inhibiting a growth of microbe according to claim 5, wherein the menthyl 3-hydroxybutanoate content is 0.0001 to 30% by weight based on the total amount of the food and beverage, the cosmetic, the grocery good, the composition for oral administration or the drug.

7. The method for inhibiting a growth of microbe according to claim 5, comprising adding menthyl 3-hydroxybutanoate and spilanthol to the food and beverage, the cosmetic, the grocery good, the composition for oral administration or the drug.

8. The method for inhibiting a growth of microbe according to claim 7, wherein the menthyl 3-hydroxybutanoate content is 0.0001 to 30% by weight based on the total amount of the food and beverage, the cosmetic, the grocery good, the composition for oral administration or the drug.
